(19) United States
(12) Patent Application Publication
(10) Pub. No.: US 2005/0101898 A1
(43) Pub. Date: May 12, 2005

(54) Orthopedic braces for the third, fourth and/or fifth metacarpals and/or phalanges

(76) Inventor: Jack E. Cohen, Green Cove Springs, FL (US)

Correspondence Address: MYERS BIGEL SIBLEY & SAJOVEC, PO BOX 37428, RALEIGH, NC, 27627, US

(21) Appl. No.: 10/979,402

(22) Filed: Nov. 2, 2004

Related U.S. Application Data
(60) Provisional application No. 60/518,573, filed on Nov. 7, 2003.

(51) Int. Cl. A61F 5/00

(52) U.S. Cl. 602/21

(57) ABSTRACT

An orthopedic brace includes: a body portion that is configured to attach to a forearm and/or wrist of a patient; a finger portion that is configured to support the fifth, fourth and/or third phalanges and restrain movement thereof; and an intermediate portion that spans the body portion and the finger portion. In this configuration, the brace can comfortably support, protect and/or restrain the injured metacarpal and/or phalange.

### RELATED APPLICATION

This application claims priority from U.S. Provisional Patent Application No. 60/518,573, filed 7 Nov. 2003, entitled “Orthopedic braces for the third, fourth and/or fifth metacarpals and/or phalanges.”

### FIELD OF THE INVENTION

The invention relates generally to orthopedic splints, and more specifically to orthopedic splints for the wrist, hand and fingers.

### BACKGROUND OF THE INVENTION

Orthopedic braces, also referred to as splints, are widely used in the treatment of various injuries and/or other medical conditions. For example, a variety of wrist and hand splints are used to assist in the treatment of various injuries or conditions of the wrist and thumb.

A conventional wrist and hand splint that can be used to assist in the treatment of various injuries or conditions of the wrist and thumb is illustrated in FIG. 1. Such splints are often used as a secondary treatment of a thumb injury; for example, such a splint may be employed after a patient suffering from a fractured thumb has worn a cast for 2-3 weeks. Typically such splints include a base portion 10 that wraps around the wrist and the distal portion of the forearm, a sleeve 12 that is fitted over the thumb, and one or more stiffening stays that can provide additional support and stability (these are not visible in FIG. 1). These splints may be made of various materials, such as cloth, vinyl, leather, foam rubber, neoprene, aluminum or other metals, and/or molded plastic material. They can be of various lengths and sizes, and may slip on and/or wrap around the extremity. They may be fastened by straps and/or other conventional connecting devices, including fabric, plastic and/or Velcro®-type straps that encircle the wrist and palm.

### SUMMARY OF THE INVENTION

The present invention is directed generally to orthopedic braces for the third, fourth and/or fifth metacarpals and/or phalanges. As a first aspect, embodiments of the present invention are directed to orthopedic braces comprising: a body portion that is configured to attach to a forearm and/or wrist of a patient; a finger portion that is configured to support the fifth, fourth and/or third phalanges and restrain movement thereof; and an intermediate portion that spans the body portion and the finger portion. In this configuration, the brace can comfortably support, protect and/or restrain the injured metacarpal and/or phalange(s).

As a second aspect, embodiments of the present invention are directed to orthopedic braces comprising: a body portion that is configured to attach to a forearm and/or wrist of a patient; a finger portion that is configured to support the fifth, fourth and/or third phalanges and restrain movement thereof, and an intermediate portion that spans the body portion and the finger portion, wherein the intermediate portion includes a stiffening stay that extends along the base of the patient's hand. This configuration can provide additional support and/or protection to the injured metacarpal and/or phalange(s).

As a third aspect, embodiments of the present invention are directed to orthopedic braces comprising: a body portion that is configured to attach to a forearm and/or wrist of a patient; a finger portion that is configured to support the fifth, fourth and/or third phalanges and restrain movement thereof, the finger portion including an adjustable strap that encircles at least the fifth phalanx; and an intermediate portion that spans the body portion and the finger portion, the intermediate portion including a void through which the patient's thumb can be received. The presence of the adjustable strap of the finger portion can facilitate donning, adjustment and removal of the brace. The void in the intermediate portion can provide additional comfort for a wearer of the device.

### BRIEF DESCRIPTION OF THE FIGURES

FIG. 1 is a perspective view of a prior art wrist and thumb splint.

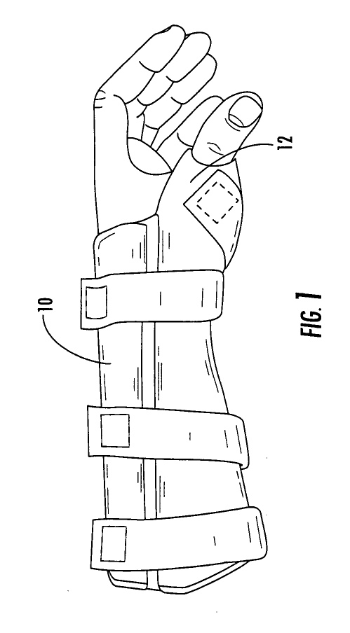

FIG. 2 is a volar view of the left hand, to illustrate an environment in which embodiments of the present invention may be employed.

FIG. 3 is a volar view of the left hand, having thereon an orthopedic brace according to various embodiments of the present invention.

FIG. 4 is a proximal end view of the brace of FIG. 3.

FIG. 5 is a bottom view of another brace according to alternative embodiments of the present invention.

FIG. 6 is a perspective view of another brace according to embodiments of the present invention worn on the left hand.

FIG. 7 is a volar view of the brace of FIG. 6.

FIG. 8 is a dorsal view of the brace of FIG. 6.

FIG. 9 is an ulnar view of the brace of FIG. 6.

### DETAILED DESCRIPTION OF EMBODIMENTS OF THE INVENTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout. Thicknesses and dimensions of some components may be exaggerated for clarity.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. As used herein, the term “and/or” includes any and all combinations of one or more of the associated listed items. Where used, the terms “attached”, “connected”, “interconnected”, “contacting”, “coupled”, “mounted” and the like can mean either direct or indirect attachment or contact between elements, unless stated otherwise.

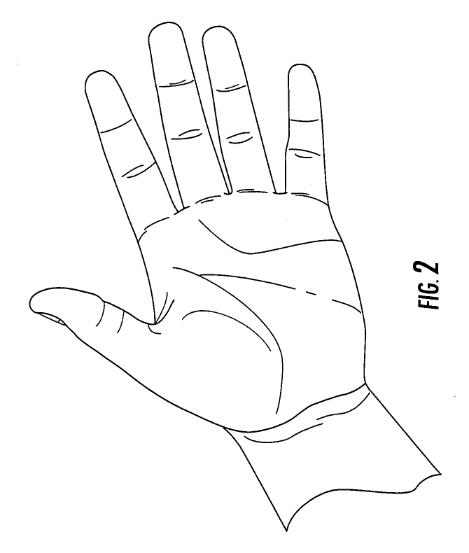

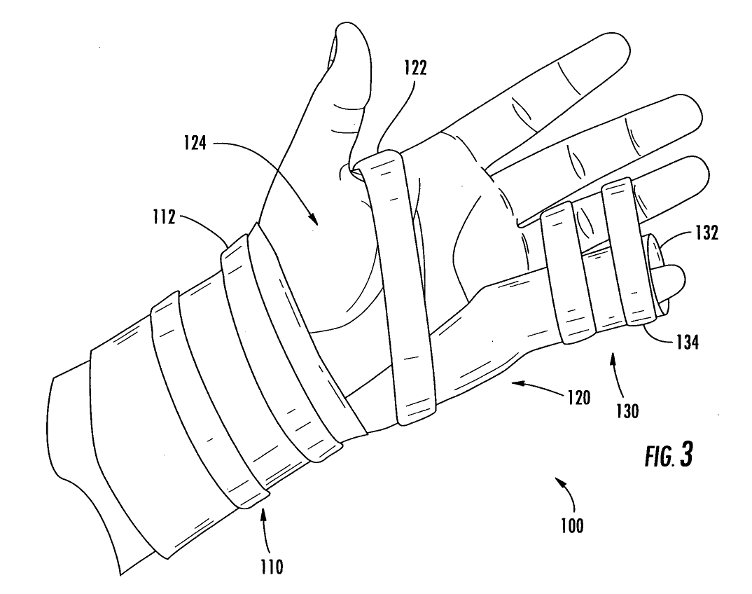

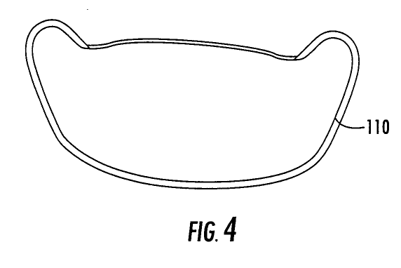

Turning now to the Figures, an orthopedic brace according to various embodiments of the present invention, designated broadly at 100, is illustrated in FIGS. 3 and 4 (FIG. 2 illustrates a hand on which the brace 100 can be used). The orthopedic brace 100 may be configured to assist in the treatment of injuries or conditions of the fifth and fourth metacarpals and associated phalanges/fingers.

As shown in FIG. 3, the orthopedic brace 100 is a removable device that can be configured to slip on and/or wrap around the hand and wrist. The illustrated embodiment includes a body portion 110 that is configured to attach to the forearm and/or wrist of the patient, a finger portion 130 that is configured to support the fifth, fourth and/or third phalanges and restrain movement thereof, and an intermediate portion 120 that is configured to span the body portion 110 and finger portion 130. In the embodiment shown in FIG. 3, the finger portion 130 is configured to support the fourth and fifth fingers. Moreover, as shown in FIG. 3, the intermediate portion 120 is configured to traverse the hand adjacent the fifth metacarpal and define a pocket for the base of the patient's hand.

Still referring to FIGS. 3 and 4, the body portion 110 may include one or more attachment straps 112 and/or other fasteners, which may be of conventional design. As an example, the attachment straps 112 may be Velcro® hook-and-loop fasteners. In other embodiments, fasteners need not be employed. For example, the body portion may be formed as an elastic sleeve, or can take a C-shaped profile with inwardly-extending lips that retain it in place.

Referring again to FIG. 3, the intermediate portion 120 spans the body portion 110 and the finger portion 130, but may include a void 124 in which the patient's thumb is received. In some embodiments the intermediate portion 120 defines a pocket for the base of the patient's hand. The intermediate portion 120 may include one or more straps 122 that are configured to encircle the hand to assist in maintain the brace 100 in place. However, in other embodiments, these straps 122 need not be included and/or other conventional supports may be used.

Referring once again to FIGS. 3 and 4, the finger portion 130 may include a sleeve 132 for the fifth phalanx. However, in other embodiments, a sleeve may be sized for multiple phalanges, or the sleeve 132 may be completely or partially omitted. In other embodiments, the sleeve 132 may also extend to the fourth or third phalanx. In yet other embodiments, the sleeve 132 need not completely encircle the phalange(s).

Referring yet again to FIG. 3, the finger portion 130 may also include one or more supportive straps or loops 134 of material to allow the fourth and/or fifth fingers to be captured by the brace 100 while not residing within the sleeve 132 of the finger portion 130. The supportive loop(s) can be further secured or fastened by additional material, which can comprise fabric, cloth, leather, vinyl, rubber, plastic, Velcro®, elastic, neoprene and/or other conventional materials. Such loops 134 can further protect the area of concern, for example the fourth and fifth metacarpals and corresponding phalanges. The loop 134 may also extend to protect the third metacarpal, or the loop 134 that surrounds the fourth and fifth metacarpals may itself, in essence, also be effective to protect the third metacarpal, depending on the pathology or condition.

In some embodiments, the brace 100 can be constructed of various materials such as fabric, cloth, leather, vinyl, rubber, plastic, Velcro®, elastic, foam, neoprene and/or other conventional materials. Certain embodiments of the brace 100 are formed of polymeric material, with the body portion 110 and the intermediate portion 120 being a unitary structure.

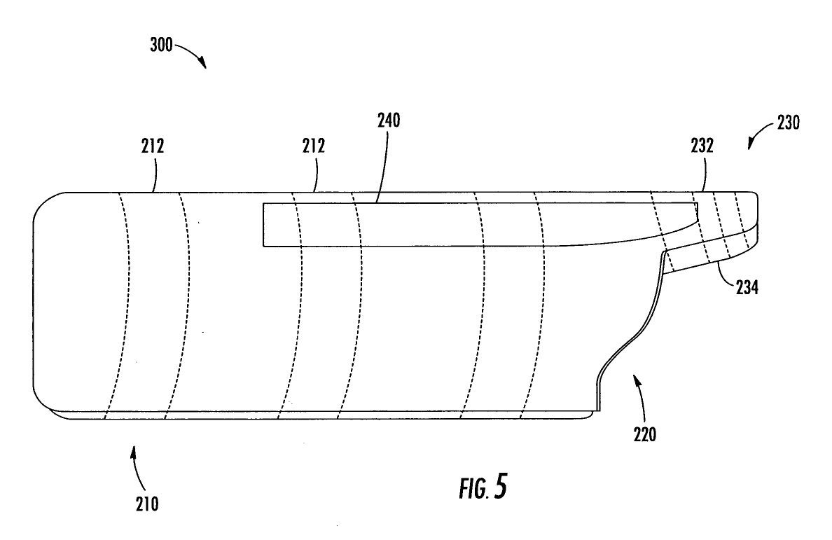

FIG. 5 is a perspective view of another orthopedic brace 300 according to other embodiments of the present invention, wherein one or more fixed and/or removable stiffening stays 240 may be incorporated to provide additional rigidity. In the illustrated embodiment, the stay 240 extends along the base of the patient's hand, but other locations for stays may also be desirable. The stay 240 may be made out of plastic, metal, aluminum and/or other conventional material. The stay 240 may be rigid and/or pliable. A stay may be incorporated into embodiments of FIGS. 3 and 4. Moreover, as was described with respect to FIG. 3, many combinations of sleeves, straps, supports and/or fasteners may be provided.

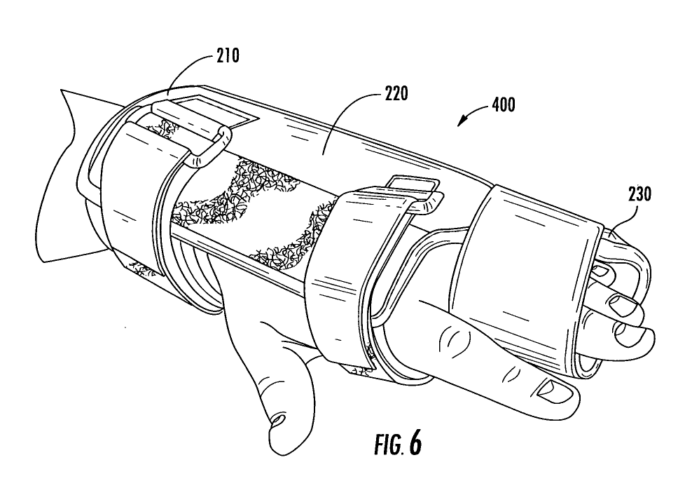

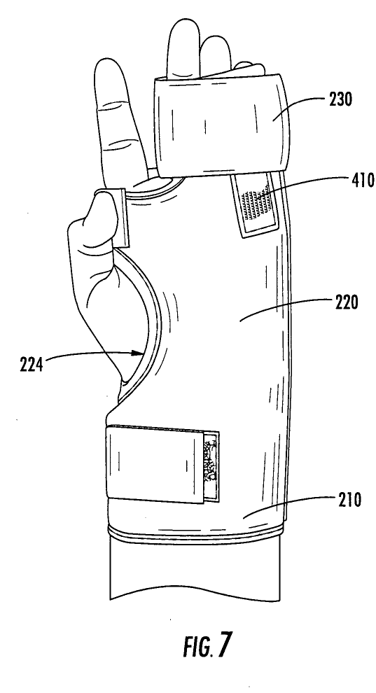

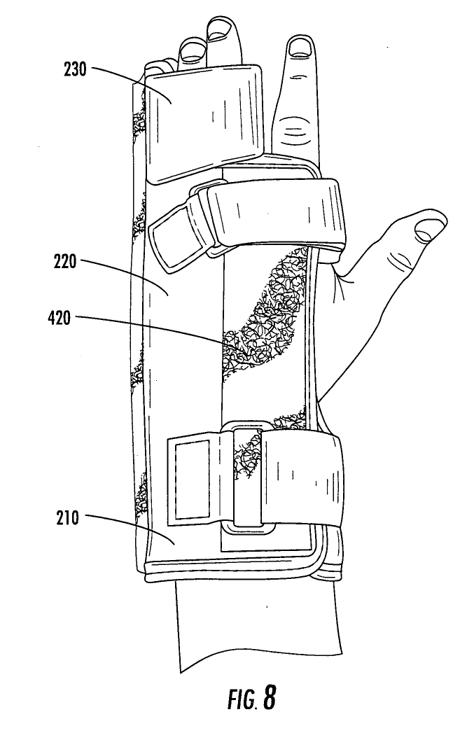

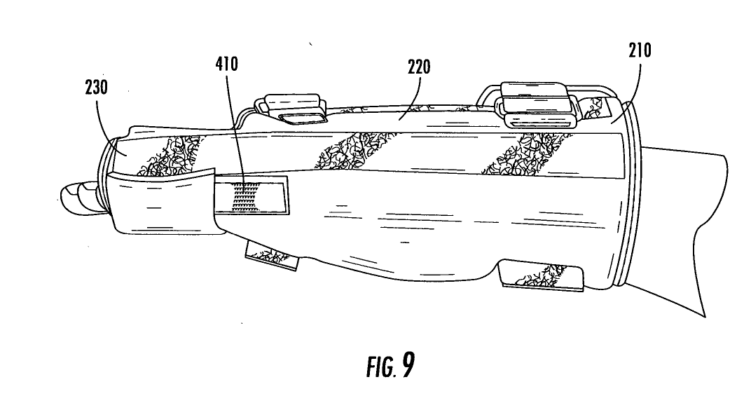

FIGS. 6-9 illustrate another orthopedic brace 400 according to other embodiments of the present invention. The brace 400 includes a body portion 210, an intermediate portion 220 with a void 224 and a finger portion 230 as described in connection with the embodiments of FIGS. 3-6. However, the finger portion 230 comprises a thick strap that can be fastened and unfastened (for example, with Velcro® fasteners) to facilitate capture of the phalanges.

In addition, the brace 400 includes a stiffening stay 410 on the front (volar) surface of the finger portion 230 and a stiffening stay 420 on the back (dorsal) surface of the intermediate portion 220. These stiffening stays 410, 420 may be included to restriction flexure of, respectively, the phalanges captured by the finger portion 230 and the wrist. As was described with respect to FIG. 3, many combinations of sleeves, straps, supports and/or fasteners may be provided.

Embodiments of the present invention may be made to accommodate a left and/or right hand. They can be made of various sizes or in a “one size fits all” configuration. They also may be of various colors. The overall construction can be woven, sewn, molded, glued and/or using other conventional materials/construction techniques that are generally used for orthopedic braces. Foam may be included for additional comfort using techniques well known to those having skill in the art.

In the drawings and specification, there have been disclosed embodiments of the invention and, although specific terms are employed, they are used in a generic and descriptive sense only and not for purposes of limitation. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. An orthopedic brace for treatment of an injury to at least one of the third, fourth or fifth metacarpal or phalange(s), comprising:
a body portion that is configured to attach to a forearm and/or wrist of a patient;
a finger portion that is configured to support the fifth, fourth and/or third phalanges and restrain movement thereof, and
an intermediate portion that spans the body portion and the finger portion.

2. The orthopedic brace defined in claim 1, wherein the intermediate portion includes a void through which the patient's thumb can be received.

3. The orthopedic brace defined in claim 1, wherein the intermediate portion defines a pocket for the base of the patient's hand.

4. The orthopedic brace defined in claim 1, further comprising attachment straps attached to the body portion that encircle the patient's forearm and/or wrist.

5. The orthopedic brace defined in claim 4, wherein the attachment straps include hook and loop fasteners.

6. The orthopedic brace defined in claim 1, wherein the finger portion comprises an adjustable strap that encircles at least the fifth phalanx.

7. The orthopedic brace defined in claim 1, wherein the body portion and intermediate portion are formed of a foamed polymeric material.

8. An orthopedic brace for treatment of an injury to at least one of the third, fourth or fifth metacarpal or phalanx, comprising:
a body portion that is configured to attach to a forearm and/or wrist of a patient;
a finger portion that is configured to support the fifth, fourth and/or third phalanges and restrain movement thereof, and
an intermediate portion that spans the body portion and the finger portion, the intermediate portion including a stiffening stay that extends along the base of the patient's hand.

9. The orthopedic brace defined in claim 8, wherein the intermediate portion defines a pocket for the base of the patient's hand.

10. The orthopedic brace defined in claim 8, wherein the intermediate portion includes a void through which the patient's thumb can be received.

11. The orthopedic brace defined in claim 8, further comprising attachment straps attached to the body portion that encircle the patient's forearm and/or wrist.

12. The orthopedic brace defined in claim 10, wherein the attachment straps include hook and loop fasteners.

13. The orthopedic brace defined in claim 8, wherein the finger portion comprises an adjustable strap that encircles at least the fifth phalanx.

14. The orthopedic brace defined in claim 8, wherein the body portion and intermediate portion are formed of a foamed polymeric material.

15. An orthopedic brace for treatment of an injury to at least one of the third, fourth or fifth metacarpal or phalanx, comprising:
a body portion that is configured to attach to a forearm and/or wrist of a patient;
a finger portion that is configured to support the fifth, fourth and/or third phalanges and restrain movement thereof, the finger portion including an adjustable strap that encircles at least the fifth phalanx; and
an intermediate portion that spans the body portion and the finger portion, the intermediate portion including a void through which the patient's thumb can be received.

16. The orthopedic brace defined in claim 15, further comprising attachment straps attached to the body portion that encircle the patient's forearm and/or wrist.

17. The orthopedic brace defined in claim 16, wherein the attachment straps include hook and loop fasteners.

18. The orthopedic brace defined in claim 15, wherein the body portion and intermediate portion are formed of a foamed polymeric material.

19. The orthopedic brace defined in claim 15, wherein the intermediate portion includes a stiffening stay that extends along the base of the patient's hand.
